(19) United States
(12) Patent Application Publication
(10) Pub. No.: US 2026/0334909 A1
(43) Pub. Date: Oct. 29, 2026

(54) METHOD AND SYSTEM FOR DETERMINING REGIONAL WEAKENING OF BLOOD VESSEL USING 2D ULTRASOUND IMAGING

(71) Applicant: VITAA MEDICAL SOLUTIONS INC., Montreal (CA)

(72) Inventors: Harold Wodlinger, Toronto (CA); Atefeh Abdolmanafi, Keswick (CA); Richard Beddoes, Montreal (CA); D. Randy Moore, Chestermere (CA); Elena di Martino, Calgary (CA)

(21) Appl. No.: 19/099,214

(22) PCT Filed: Jul. 28, 2023
(86) PCT No.: PCT/IB2023/057708

§ 371 Date: Jan. 28, 2025

Related U.S. Application Data
(60) Provisional application No. 63/369,813, filed on Jul. 29, 2022.

(51) Int. Cl. A61B 8/08; A61B 8/00; A61B 8/06

(52) U.S. Cl. CPC A61B 8/0891; A61B 8/06; A61B 8/488; A61B 8/5223

(57) ABSTRACT

A method and system for determining regional weakening (RW) values in a blood vessel using ultrasound imaging. A set of acquired images of a portion of a body of a given patient time are received. A set of measurements representative of movements of tissues of the blood vessel are received, the set of measurements having been determined based on the set of images. Movements of the wall and the lumen are at least partially indicative of strain. Blood velocity values in the blood vessel are determined based on Doppler images, and a RW parameter indicative of a state of weakening in regions of the blood vessel is determined based on at least the set of measurements, thrombus load and blood velocity values. A map showing a circumference of the blood vessel including regional weakening and strain values may be displayed.

### CROSS-REFERENCE TO RELATED APPLICATIONS

The present application claims priority on U.S. Provisional Patent Application No. 63/369,813 filed on Jul. 29, 2022.

### FIELD

The present technology pertains to the field of medical imaging. More precisely, the present technology relates to methods and systems for performing regional weakening (RW) analysis of a blood vessel using two-dimensional (2D) ultrasound imaging.

### BACKGROUND

Regional weakening (RW) analysis, which may also be referred to as regional rupture potential (RRP), enables performing assessment of vessels based on parameters that correlate with the local weakening, expansion and rupture of the vessel and provides a rationale for clinical decisions by performing calculations solely based on images acquired by a medical imaging apparatus. For an aorta, regional weakening is referred to as regional aortic weakening (RAW) analysis, which identifies areas of weakness of the wall of the aorta, including aortic aneurysms, in order to predict growth and potential rupture, and to allow physicians to treat the right patient at the right time. RAW analysis is currently performed using dynamic computed tomography (CT) and magnetic resonance imaging (MRI) scans. These scans are segmented to produce accurate three-dimensional (3D) and four-dimensional (4D) models of the aorta, which are analysed to measure local strain, wall sheer stress, and thrombus load. These three measurements are combined to produce the RAW index. Physicians are presented with detailed aortic maps showing these variables and showing the RAW index. Regional weakening values of other blood vessels such as common iliac arteries and visceral arteries can be determined in a similar manner when imaging resolution is sufficient.

RAW analysis using ultrasound imaging would be preferred to either CT or MRI imaging because it would be more convenient for both the patient and the physician, is less expensive, and does not use ionizing radiation or toxic contrast chemicals. Ultrasound imaging has higher resolution than either CT or potentially toxic MRI, which will allow accurate measurement of aortic wall thickness. However, RAW analysis using ultrasound imaging is a technical challenge compared to CT and MRI imaging. Ultrasound imaging does not produce evenly spaced image slices like CT and MRI, making it difficult to produce accurate 3D and 4D models of the aorta. The ultrasound transducer is handheld, so the angles of the image relative to the body and the aorta are variable, again making it difficult to create an accurate model. In addition, ultrasound images have numerous artifacts including reflections, shadowing, refraction, etc., and the resolution within the ultrasound image varies with the depth of the structure.

### SUMMARY

It is an object of the present technology to ameliorate at least some of the inconveniences present in the prior art. One or more embodiments of the present technology may provide and/or broaden the scope of approaches to and/or methods of achieving the aims and objects of the present technology.

Developers of the present technology have appreciated that three-dimensional ultrasound imaging is available on many high-end ultrasound scanners, however these scanners are extremely expensive and currently have poor resolution and small fields of view. Regional weakening analysis requires 4D imaging (3 spatial dimensions plus time to see movement), and the field of view of 4D imaging using ultrasound may be too small to be useful at reasonable frame rates in some contexts.

Developers of the present technology have theorized that it is possible to measure strain and intraluminal thrombus (ILT), and to calculate regional weakening indices using standard 2D ultrasound scanners that are commonly found in medical centres.

Developers of the present technology propose calculating a regional weakening (RW) index without the step of building a 3D model of the blood vessel (e.g. aorta). The RW index would be calculated only for the axial image that is generated via the ultrasound probe and displayed on the screen. For example, the user or operator would move the ultrasound probe up and down the aorta examining areas of interest, the RW index would be calculated and displayed, and diameters would be automatically measured and reported to the user. Then, minimum, maximum, and equivalent circle diameters could be calculated and would provide a measure of eccentricity.

The present technology will enable vascular surgeons to perform regional weakening analysis in their office without having to send the patient for a dynamic CT or MRI scan, thereby avoiding the radiation associated with CT scans, and avoiding potentially toxic contrast agents. The present technology fits existing workflows, as well as being far less time consuming and expensive. Analysis could be performed in the cloud, the software could potentially be integrated within an ultrasound scanner, or the software could be run on a computer connected to the video output of the medical imaging apparatus.

Ultrasound imaging has higher resolution than either CT or MRI, which enables accurate measurement of aortic wall thickness. Having the local thickness will enable the surgeon to assess a local material stiffness (based on strain and blood pressure). In addition, this will enable refinement of the regional weakening map to compensate for the loss of accuracy and 3D assessment.

Ultrasound imaging also has higher temporal resolution than either CT or MRI. Higher temporal resolution might allow assessment of viscoelasticity in the wall (and in the thrombus). Viscoelastic properties, in particular energy loss, have been linked to wall degeneration in the thoracic aorta (see paper by Chung J, Lachapelle K, Wener E, Cartier R, De Varennes B, Fraser R, Leask RL. Energy loss, a novel biomechanical parameter, correlates with aortic aneurysm size and histopathologic findings. J Thorac Cardiovasc Surg. 2014 Sep; 148(3): 1082-8; discussion 1088-9. doi: 10.1016/j.jtcvs.2014.06.021. Epub 2014 Jun 13. PMID: 25129601) further, it was demonstrated that in the abdominal aorta, a high RAW corresponds to higher energy loss, (see paper Forneris, A., Nightingale, M.; Ismaguilova, A.; Sigaeva, T., Neave, L.; Bromley, A.; Moore, R.D.; Di Martino, E.S. Heterogeneity of Ex Vivo and In Vivo Properties along the Length of the Abdominal Aortic Aneurysm. Appl. Sci. 2021, 11, 3485. https://doi.org/10.3390/app11083485).

Thus, one or more embodiments of the present technology are directed to a method of and a system for determining regional weakening of a blood vessel using 2D ultrasound imaging.

In accordance with a broad aspect of the present technology, there is provided a method for determining regional weakening parameters of a blood vessel of a given patient, the method being executed by at least one processing device. The method comprises: receiving a set of images of a portion of a body of the given patient having been acquired over time using an ultrasound imaging apparatus, the set of images comprises Doppler images, receiving a set of measurements representative of movements of a wall and lumen of the blood vessel, the set of measurements having been determined based on the set of images, determining, based on the Doppler images in the set of images, blood velocity values in the blood vessel, generating, based on at least the set of measurements and the blood velocity values, a regional weakening parameter indicative of a state of weakening in regions of the blood vessel, and outputting the regional weakening parameter.

In one or more embodiments of the method, the method further comprises, prior to said generating, based on at least the set of measurements and the blood velocity values, the regional weakening parameter indicative of a state of weakening in regions of the blood vessel: determining, by analyzing the set of images over time based on the set of measurements, a radial deformation in the blood vessel, the radial deformation being at least partially indicative of strain in the blood vessel, said generating the regional weakening parameter is based on the radial deformation in the blood vessel.

In one or more embodiments of the method, the method further comprises, after said receiving the set of images: detecting, for the set of images over time, respective wall and lumen positions in the blood vessel, and calculating, based on the respective wall and lumen positions over time, a movement of the wall and the lumen of the blood vessel to thereby receive the set of measurements.

In one or more embodiments of the method, said calculating, based on the respective wall and lumen positions over time, the movement of the wall and the lumen of the blood vessel comprises: determining, based on the positions of the wall and the lumen, a thrombus thickness, and said generating the regional weakening parameter is based on the thrombus thickness.

In one or more embodiments of the method, the thrombus thickness of 0 is indicative of absence of a thrombus in the blood vessel.

In one or more embodiments of the method, the blood vessel comprises at least one of: an aorta, common iliac arteries, and visceral arteries.

In one or more embodiments of the method, the method further comprises generating a regional weakening map based on the regional weakening parameter and the set of images of the blood vessel, and outputting the regional weakening map, the regional weakening map showing a circumference of the blood vessel comprises regional weakening values.

In one or more embodiments of the method, said detecting, for the set of images over time, respective wall and lumen positions in the blood vessel comprises: segmenting, using a trained segmentation model on the set of images, an outside wall, an inside wall, and the lumen of the blood vessel.

In one or more embodiments of the method, the method further comprises: determining minimum, maximum, equivalent circle diameters and eccentricity of each of an outside wall diameter, an inside wall diameter and a lumen diameter.

In one or more embodiments of the method, the trained segmentation model comprises a fully convolutional neural network (FCN).

In one or more embodiments of the method, the method further comprises, prior to said receiving the set of images of the blood vessel: receiving, a plurality of images of the given patient having been acquired using the ultrasound imaging apparatus, the plurality of images including at least one Doppler image, generating, using a trained resolution enhancing machine learning (ML) model on the plurality of images, a plurality of high resolution (HR) images of the blood vessel, a resolution of the plurality of HR images being above a resolution of the plurality of images, and denoising, using a trained ML denoising model, the plurality of HR images to obtain the set of images.

In one or more embodiments of the method, the trained resolution enhancing ML model comprises a very deep super-resolution (VDSR) neural network, and the trained denoising ML model comprises a cycle-consistent generative adversarial network (CycleGAN).

In one or more embodiments of the method, the method further comprises: generating a strain map based on the radial deformation values and the set of images of the blood vessel, and outputting the strain map, the strain map showing a circumference of the blood vessel comprises strain values.

In one or more embodiments of the method, the method further comprises determining a viscoelasticity of the blood vessel wall.

In one or more embodiments of the method, the method further comprises determining a viscoelasticity of the thrombus.

In one or more embodiments of the method, the at least one processing device is operatively connected to the ultrasound imaging apparatus.

In one or more embodiments of the method, the method further comprises: displaying, on a display operatively connected to the at least one processing device, the regional weakening map.

In accordance with a broad aspect of the present technology, there is provided a system for determining regional weakening (RW) of a blood vessel of a given patient. The system comprises: at least one processing device, and a non-transitory storage medium operatively connected to the at least one processing device, the non-transitory storage medium storing computer-readable instructions. The processor, upon executing the computer-readable instructions, being configured for: receiving a set of images of a portion of a body of the given patient having been acquired over time using an ultrasound imaging apparatus, the set of images comprises Doppler images, receiving a set of measurements representative of movements of a wall and lumen of the blood vessel, the set of measurements having been determined based on the set of images, determining, based on the Doppler images in the set of images, blood velocity values in the blood vessel, generating, based on at least the set of measurements and the blood velocity values, a regional weakening parameter indicative of a state of weakening in regions of the blood vessel, and outputting the regional weakening parameter.

In one or more embodiments of the system, the at least one processing device is further configured for, prior to said generating, based on at least the set of measurements and the blood velocity values, the regional weakening parameter indicative of a state of weakening in regions of the blood vessel: determining, by analyzing the set of images over time based on the set of measurements, a radial deformation in the blood vessel, the radial deformation being at least partially indicative of strain in the blood vessel, said generating the regional weakening parameter is based on the radial deformation in the blood vessel.

In one or more embodiments of the system, the at least one processing device is further configured for, after said receiving the set of images: detecting, for the set of images over time, respective wall and lumen positions in the blood vessel, and calculating, based on the respective wall and lumen positions over time, a movement of the wall and the lumen of the blood vessel to thereby receive the set of measurements.

In one or more embodiments of the system, said calculating, based on the respective wall and lumen positions over time, the movement of the wall and the lumen of the blood vessel comprises: determining, based on the positions of the wall and the lumen, a thrombus thickness, and said generating the regional weakening parameter is based on the thrombus thickness.

In one or more embodiments of the system, the thrombus thickness of 0 is indicative of absence of a thrombus in the blood vessel.

In one or more embodiments of the system, the blood vessel comprises at least one of: an aorta, common iliac arteries, and visceral arteries.

In one or more embodiments of the system, the at least one processing device is further configured for: generating a regional weakening map based on the regional weakening parameter and the set of images of the blood vessel, and outputting the regional weakening map, the regional weakening map showing a circumference of the blood vessel comprises regional weakening values.

In one or more embodiments of the system, said detecting, for the set of images over time, respective wall and lumen positions in the blood vessel comprises: segmenting, using a trained segmentation model on the set of images, an outside wall, an inside wall, and the lumen of the blood vessel.

In one or more embodiments of the system, the at least one processing device is further configured for: determining minimum, maximum, equivalent circle diameters and eccentricity of each of an outside wall diameter, an inside wall diameter and a lumen diameter.

In one or more embodiments of the system, the trained segmentation model comprises a fully convolutional neural network (FCN).

In one or more embodiments of the system, the at least one processing device is further configured for, prior to said receiving the set of images of the blood vessel: receiving, a plurality of images of the given patient having been acquired using the ultrasound imaging apparatus, the plurality of images including at least one Doppler image, generating, using a trained resolution enhancing machine learning (ML) model on the plurality of images, a plurality of high resolution (HR) images of the blood vessel, a resolution of the plurality of HR images being above a resolution of the plurality of images, and denoising, using a trained ML denoising model, the plurality of HR images to obtain the set of images.

In one or more embodiments of the system, the trained resolution enhancing ML model comprises a very deep super-resolution (VDSR) neural network, and the trained denoising ML model comprises a cycle-consistent generative adversarial network (CycleGAN).

In one or more embodiments of the system, the at least one processing device is further configured for: generating a strain map based on the radial deformation values and the set of images of the blood vessel, and outputting the strain map, the strain map showing a circumference of the blood vessel comprises strain values.

In one or more embodiments of the system, the at least one processing device is further configured for determining a viscoelasticity of the blood vessel wall.

In one or more embodiments of the system, the at least one processing device is further configured for determining a viscoelasticity of the thrombus.

In one or more embodiments of the system, the at least one processing device is operatively connected to the ultrasound imaging apparatus.

In one or more embodiments of the system, the at least one processing device is further configured for: displaying, on a display operatively connected to the at least one processing device, the regional weakening map.

### Terms and Definitions

In the context of the present specification, a “server” is a computer program that is running on appropriate hardware and is capable of receiving requests (e.g., from electronic devices) over a network (e.g., a communication network), and carrying out those requests, or causing those requests to be carried out. The hardware may be one physical computer or one physical computer system, but neither is required to be the case with respect to the present technology. In the present context, the use of the expression “a server” is not intended to mean that every task (e.g., received instructions or requests) or any particular task will have been received, carried out, or caused to be carried out, by the same server (i.e., the same software and/or hardware); it is intended to mean that any number of software elements or hardware devices may be involved in receiving/sending, carrying out or causing to be carried out any task or request, or the consequences of any task or request; and all of this software and hardware may be one server or multiple servers, both of which are included within the expressions “at least one server” and “a server”.

In the context of the present specification, “electronic device” is any computing apparatus or computer hardware that is capable of running software appropriate to the relevant task at hand. Thus, some (non-limiting) examples of electronic devices include general purpose personal computers (desktops, laptops, netbooks, etc.), mobile computing devices, smartphones, and tablets, and network equipment such as routers, switches, and gateways. It should be noted that an electronic device in the present context is not precluded from acting as a server to other electronic devices. The use of the expression “an electronic device” does not preclude multiple electronic devices being used in receiving/sending, carrying out or causing to be carried out any task or request, or the consequences of any task or request, or steps of any method described herein. In the context of the present specification, a “client device” refers to any of a range of end-user client electronic devices, associated with a user, such as personal computers, tablets, smartphones, and the like.

In the context of the present specification, unless expressly provided otherwise, a computer system may refer, but is not limited to, an “electronic device”, a “computing device”, an “operation system”, a “system”, a “computer-based system”, a “computer system”, a “network system”, a “network device”, a “controller unit”, a “monitoring device”, a “control device”, a “server”, and/or any combination thereof appropriate to the relevant task at hand.

In the context of the present specification, the expression “computer readable storage medium” (also referred to as “storage medium” and “storage”) is intended to include non-transitory media of any nature and kind whatsoever, including without limitation RAM, ROM, disks (CD-ROMs, DVDs, floppy disks, hard drivers, etc.), USB keys, solid state-drives, tape drives, etc. A plurality of components may be combined to form the computer information storage media, including two or more media components of a same type and/or two or more media components of different types.

In the context of the present specification, a “database” is any structured collection of data, irrespective of its particular structure, the database management software, or the computer hardware on which the data is stored, implemented or otherwise rendered available for use. A database may reside on the same hardware as the process that stores or makes use of the information stored in the database or it may reside on separate hardware, such as a dedicated server or plurality of servers.

In the context of the present specification, the expression “information” includes information of any nature or kind whatsoever capable of being stored in a database. Thus, information includes, but is not limited to audiovisual works (images, movies, sound records, presentations etc.), data (location data, numerical data, etc.), text (opinions, comments, questions, messages, etc.), documents, spreadsheets, lists of words, etc.

In the context of the present specification, unless expressly provided otherwise, an “indication” of an information element may be the information element itself or a pointer, reference, link, or other indirect mechanism enabling the recipient of the indication to locate a network, memory, database, or other computer-readable medium location from which the information element may be retrieved. For example, an indication of a document could include the document itself (i.e. its contents), or it could be a unique document descriptor identifying a file with respect to a particular file system, or some other means of directing the recipient of the indication to a network location, memory address, database table, or other location where the file may be accessed. As one skilled in the art would recognize, the degree of precision required in such an indication depends on the extent of any prior understanding about the interpretation to be given to information being exchanged as between the sender and the recipient of the indication. For example, if it is understood prior to a communication between a sender and a recipient that an indication of an information element will take the form of a database key for an entry in a particular table of a predetermined database containing the information element, then the sending of the database key is all that is required to effectively convey the information element to the recipient, even though the information element itself was not transmitted as between the sender and the recipient of the indication.

In the context of the present specification, the expression “communication network” is intended to include a telecommunications network such as a computer network, the Internet, a telephone network, a Telex network, a TCP/IP data network (e.g., a WAN network, a LAN network, etc.), and the like. The term “communication network” includes a wired network or direct-wired connection, and wireless media such as acoustic, radio frequency (RF), infrared and other wireless media, as well as combinations of any of the above.

In the context of the present specification, the words “first”, “second”, “third”, etc. have been used as adjectives only for the purpose of allowing for distinction between the nouns that they modify from one another, and not for the purpose of describing any particular relationship between those nouns. Thus, for example, it should be understood that, the use of the terms “first server” and “third server” is not intended to imply any particular order, type, chronology, hierarchy or ranking (for example) of/between the servers, nor is their use (by itself) intended to imply that any “second server” must necessarily exist in any given situation. Further, as is discussed herein in other contexts, reference to a “first” element and a “second” element does not preclude the two elements from being the same actual real-world element. Thus, for example, in some instances, a “first” server and a “second” server may be the same software and/or hardware, in other cases they may be different software and/or hardware.

Implementations of the present technology each have at least one of the above-mentioned objects and/or aspects, but do not necessarily have all of them. It should be understood that some aspects of the present technology that have resulted from attempting to attain the above-mentioned object may not satisfy this object and/or may satisfy other objects not specifically recited herein.

Additional and/or alternative features, aspects and advantages of implementations of the present technology will become apparent from the following description, the accompanying drawings and the appended claims.

### BRIEF DESCRIPTION OF THE DRAWINGS

For a better understanding of the present technology, as well as other aspects and further features thereof, reference is made to the following description which is to be used in conjunction with the accompanying drawings, where:

FIG. 1 illustrates a schematic diagram of an electronic device in accordance with one or more non-limiting embodiments of the present technology.

FIG. 2 illustrates a schematic diagram of a communication system in accordance with one or more non-limiting embodiments of the present technology.

FIG. 3 illustrates a schematic diagram of regional weakening (RW) map generation procedure in accordance with one or more non-limiting embodiments of the present technology.

FIG. 4 illustrates an example of an axial ultrasound image of a body of a patient in accordance with one or more non-limiting embodiments of the present technology.

FIG. 5 illustrates an example of segmentation in the axial ultrasound image of FIG. 4 including borders of the outside wall, inside wall, lumen and presence of a thrombus in accordance with one or more non-limiting embodiments of the present technology.

FIG. 6 illustrates a simulated Doppler ultrasound image of the segmented aorta of FIG. 5 in accordance with one or more non-limiting embodiments of the present technology.

FIG. 7 illustrates an example of an ultrasound image of the aorta in accordance with one or more non-limiting embodiments of the present technology.

FIG. 8 illustrates an example of an ultrasound image of the aorta in accordance with one or more non-limiting embodiments of the present technology.

FIG. 9 illustrates a flowchart of a method for determining regional weakening (RW) using ultrasound imaging, the method being executed in accordance with one or more non-limiting embodiments of the present technology.

### DETAILED DESCRIPTION

The examples and conditional language recited herein are principally intended to aid the reader in understanding the principles of the present technology and not to limit its scope to such specifically recited examples and conditions. It will be appreciated that those skilled in the art may devise various arrangements which, although not explicitly described or shown herein, nonetheless embody the principles of the present technology and are included within its spirit and scope.

Furthermore, as an aid to understanding, the following description may describe relatively simplified implementations of the present technology. As persons skilled in the art would understand, various implementations of the present technology may be of a greater complexity.

In some cases, what are believed to be helpful examples of modifications to the present technology may also be set forth. This is done merely as an aid to understanding, and, again, not to define the scope or set forth the bounds of the present technology. These modifications are not an exhaustive list, and a person skilled in the art may make other modifications while nonetheless remaining within the scope of the present technology. Further, where no examples of modifications have been set forth, it should not be interpreted that no modifications are possible and/or that what is described is the sole manner of implementing that element of the present technology.

Moreover, all statements herein reciting principles, aspects, and implementations of the present technology, as well as specific examples thereof, are intended to encompass both structural and functional equivalents thereof, whether they are currently known or developed in the future. Thus, for example, it will be appreciated by those skilled in the art that any block diagrams herein represent conceptual views of illustrative circuitry embodying the principles of the present technology. Similarly, it will be appreciated that any flowcharts, flow diagrams, state transition diagrams, pseudo-code, and the like represent various processes which may be substantially represented in computer-readable media and so executed by a computer or processor, whether or not such computer or processor is explicitly shown.

The functions of the various elements shown in the figures, including any functional block labeled as a “processor” or a “graphics processing unit”, may be provided through the use of dedicated hardware as well as hardware capable of executing software in association with appropriate software. When provided by a processor, the functions may be provided by a single dedicated processor, by a single shared processor, or by a plurality of individual processors, some of which may be shared. In some non-limiting embodiments of the present technology, the processor may be a general-purpose processor, such as a central processing unit (CPU) or a processor dedicated to a specific purpose, such as a graphics processing unit (GPU). Moreover, explicit use of the term “processor” or “controller” should not be construed to refer exclusively to hardware capable of executing software, and may implicitly include, without limitation, digital signal processor (DSP) hardware, network processor, application specific integrated circuit (ASIC), field programmable gate array (FPGA), read-only memory (ROM) for storing software, random access memory (RAM), and non-volatile storage. Other hardware, conventional and/or custom, may also be included.

Software modules, or simply modules which are implied to be software, may be represented herein as any combination of flowchart elements or other elements indicating performance of process steps and/or textual description. Such modules may be executed by hardware that is expressly or implicitly shown.

With these fundamentals in place, we will now consider some non-limiting examples to illustrate various implementations of aspects of the present technology.

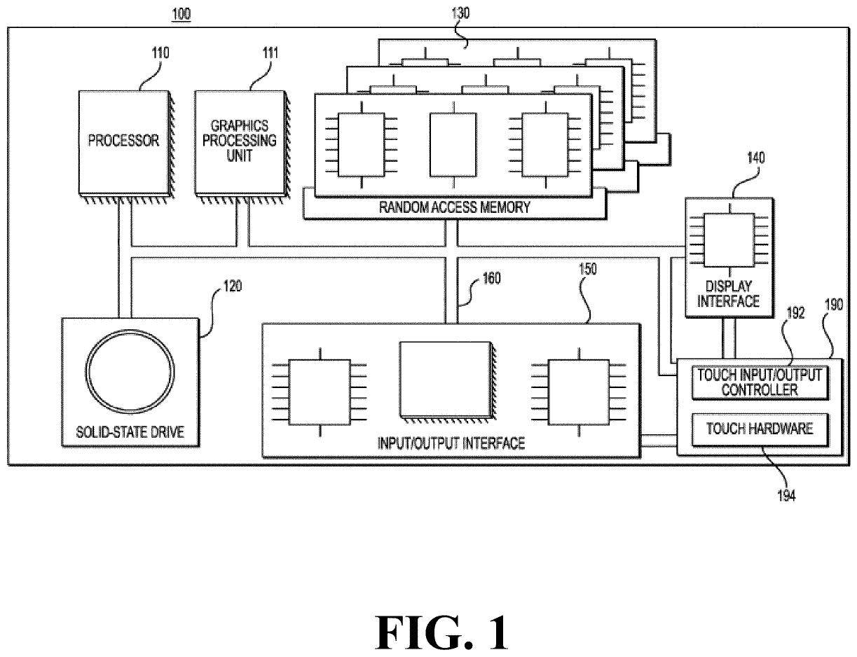

With reference to FIG. 1, there is illustrated a schematic diagram of an electronic device 100 suitable for use with some non-limiting embodiments of the present technology.

### Electronic Device

The electronic device 100 comprises various hardware components including one or more single or multi-core processors collectively represented by processor 110, a graphics processing unit (GPU) 111, a solid-state drive 120, a random-access memory 130, a display interface 140, and an input/output interface 150.

Communication between the various components of the electronic device 100 may be enabled by one or more internal and/or external buses 160 (e.g. a PCI bus, universal serial bus, IEEE 1394 “Firewire” bus, SCSI bus, Serial-ATA bus, etc.), to which the various hardware components are electronically coupled.

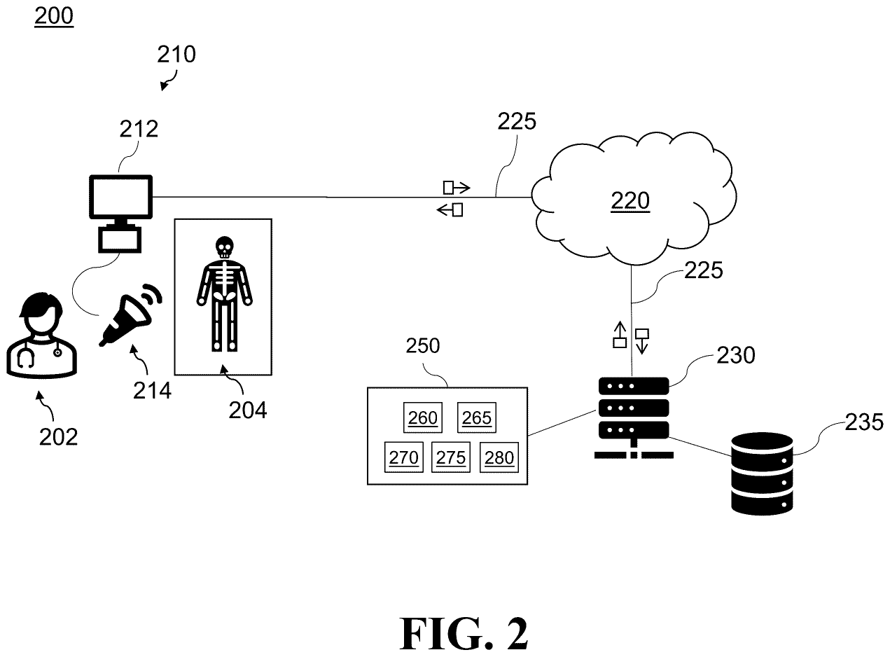

The input/output interface 150 may be coupled to a touchscreen 190 and/or to the one or more internal and/or external buses 160. The touchscreen 190 may be part of the display. In some embodiments, the touchscreen 190 is the display. The touchscreen 190 may equally be referred to as a screen 190. In the embodiments illustrated in FIG. 2, the touchscreen 190 comprises touch hardware 194 (e.g., pressure-sensitive cells embedded in a layer of a display allowing detection of a physical interaction between a user and the display) and a touch input/output controller 192 allowing communication with the display interface 140 and/or the one or more internal and/or external buses 160. In some embodiments, the input/output interface 150 may be connected to a keyboard (not shown), a mouse (not shown) or a trackpad (not shown) allowing the user to interact with the electronic device 100 in addition or in replacement of the touchscreen 190.

According to implementations of the present technology, the solid-state drive 120 stores program instructions suitable for being loaded into the random-access memory 130 and executed by the processor 110 and/or the GPU 111 for performing regional weakening (RW) analysis of a blood vessel using 2D ultrasound imaging. For example, the program instructions may be part of a library or an application.

The electronic device 100 may be implemented in the form of a server, a desktop computer, a laptop computer, a tablet, a smartphone, a personal digital assistant or any device that may be configured to implement the present technology, as it may be understood by a person skilled in the art.

### System

Referring to FIG. 2, there is shown a schematic diagram of a communication system 200, which will be referred to as the system 200, the system 200 being suitable for implementing non-limiting embodiments of the present technology. It is to be expressly understood that the system 200 as illustrated is merely an illustrative implementation of the present technology. Thus, the description thereof that follows is intended to be only a description of illustrative examples of the present technology. This description is not intended to define the scope or set forth the bounds of the present technology. In some cases, what are believed to be helpful examples of modifications to the system 200 may also be set forth below. This is done merely as an aid to understanding, and, again, not to define the scope or set forth the bounds of the present technology. These modifications are not an exhaustive list, and, as a person skilled in the art would understand, other modifications are likely possible. Further, where this has not been done (i.e., where no examples of modifications have been set forth), it should not be interpreted that no modifications are possible and/or that what is described is the sole manner of implementing that element of the present technology. As a person skilled in the art would understand, this is likely not the case. In addition it is to be understood that the system 200 may provide in certain instances simple implementations of the present technology, and that where such is the case they have been presented in this manner as an aid to understanding. As persons skilled in the art would understand, various implementations of the present technology may be of a greater complexity.

The system 200 comprises inter alia a medical imaging apparatus 210, a server 230 and a database 235 coupled over a communications network 220 via respective communication links 225 (not separately numbered).

In one or more embodiments, at least a portion of the system 200 implements the Picture Archiving and Communication System (PACS) technology.

The medical imaging apparatus 210 is operated by a user 202 (e.g., physician, ultrasonographer or technician) to acquire medical images of portions of the body of a given patient 204.

### Medical Imaging Apparatus

The medical imaging apparatus 210 is an ultrasound imaging apparatus comprising a housing 212 connected to an ultrasound probe or transducer 214. The medical imaging apparatus 210 is configured to generate images of the inner body by using high frequency sound waves transmitted and received by the probe 214.

In one or more embodiments, the housing 212 may include components such as one or more processing devices, storage mediums, displays, wired and/or wireless communication interfaces for connecting to the probe 214 and for processing reflected ultrasound energy that is received by the probe 214 to produce and display an image of the scanned anatomical region. In some embodiments, the housing 212 may include at least a portion of the components of the electronic device 100 of FIG. 1. Thus, images and other type of data may be stored in one or more storage mediums of the housing 212 and/or transmitted to other electronic devices (e.g., server 230). A display may provide feedback to the user 202 when acquiring images via the probe 214 by displaying the images, acquisition parameters, patient information and other additional information as known in the art.

The probe 214 includes one or more ultrasound transceiver elements and one or more transducer elements to convert electrical signals and transmit ultrasound signals toward a patient's area of interest (e.g., a blood vessel, organ, joint, etc.) and receive acoustic reflections or echoes generated by internal structures/tissue within the anatomical portion which may be converted back by the probe 214 into electrical signals and transmitted for conversion into image data.

In one or more other embodiments, the probe 214 may integrate the functionality of at least some of the components of the housing 212 and include a processing device (e.g., microprocessor), firmware, and/or software associated with the processing device to operably control the probe 214, and to process the reflected ultrasound energy to generate the ultrasound image. The probe 214 may include a display (such as a liquid crystal display (LCD), light emitting diode (LED) based display, or other type of display that provides text and/or image data to a user) and/or the images may be transmitted for display on a mobile device such as a smartphone or a tablet.

In use, the user 202 may position the probe 214 over a portion of the body comprising a target blood vessel, such as, but not limited to, the abdominal aorta to obtain an image of the abdominal aorta.

The medical imaging apparatus 210 is configured to inter alia: (i) acquire of a blood vessel of a given patient 204 during period of time according to respective acquisition parameters; and (ii) acquire Doppler images of the blood vessel of the given patient 204 according to respective acquisition parameters.

In the context of the present technology, the medical imaging apparatus 210 is configured to acquire images of blood vessels such as aortas and/or iliac arteries and/or visceral arteries. The medical imaging apparatus 210 may be configured with specific acquisition parameters for acquiring images of the patient comprising the target blood vessels. The acquisition parameters (e.g., scan mode, amplitude, frequency and duration of the pulses emitted from the probe 214) may be specified by the user 202 via an input/output interface of the medical imaging apparatus 210 such as a keyboard, cursor, and/or touchscreen. As a non-limiting example, acquisition parameters include choice of transducer (curvilinear phased array, 2-5 MHz); optimization of contrast and brightness (referred to as “window” and “level”), and optimization of ultrasound gain and focus zones.

The user 202 may further perform additional operations using the input/output interface, such as measurements in images, as well as image processing operations (e.g., filtering, enhancement, smoothing, display format), calculations and annotations that may be displayed on a display screen of the medical imaging apparatus 210.

In some embodiments, the medical imaging apparatus 210 is configured to perform Doppler ultrasonography. It will be appreciated that Doppler ultrasonography uses the Doppler effect to perform imaging of the movement of tissues and body fluids and their relative velocity to the probe.

The medical imaging apparatus 210 is configured to perform color flow Doppler imaging to determine blood flow velocities in the blood vessel (e.g., aorta). The medical imaging apparatus 210 may be configured to generate multicolored signals (e.g., with red for flows towards probe and blue away for flow away from probe) when performing Doppler imaging.

In one or more alternative embodiments, the medical imaging apparatus 210 may include or may be connected to a workstation computer (not illustrated) for inter alia control of acquisition parameters and image data transmission.

In one or more embodiments, a workstation computer may be provided together with the medical imaging apparatus 210, i.e. in the housing 212. In one or more other embodiments, the workstation computer may be implemented as a mobile device such as a smartphone or a tablet.

In one or more embodiments, the medical imaging apparatus 210 is part of a Picture Archiving and Communication System (PACS) for storing and retrieving medical images together with other electronic devices such as the server 230.

### Server

The server 230 is configured to inter alia: (i) receive ultrasound images including Doppler images; (ii) perform, using the ultrasound images, thickness measurements, flow disturbance measurements, and deformation measurements; (iii) generate, based on the ultrasound images, the thickness measurements, the flow disturbance measurements, and the deformation measurements, a radial strain values parameter or index; (iv) generate, based on the ultrasound images, the thickness measurements, the flow disturbance measurements, the deformation measurements, and the radial strain index, a regional weakness (RW) parameter or index; and (v) generate one of a radial strain map based on the radial strain index and/or a RW map based on the RW index of the circumference of the wall of the aorta.

How the server 230 is configured to do so will be explained in more detail herein below.

The server 230 can be implemented as a conventional computer server and may comprise some or all of the components of the electronic device 100 illustrated in FIG. 2. In an example of one or more embodiments of the present technology, the server 230 can be implemented as a Dell™ PowerEdge™ Server running the Microsoft™ Windows Server™ operating system. Needless to say, the server 230 can be implemented in any other suitable hardware and/or software and/or firmware or a combination thereof. In the illustrated non-limiting embodiment of present technology, the server 230 is a single server. In alternative non-limiting embodiments of the present technology, the functionality of the server 230 may be distributed and may be implemented via multiple servers (not illustrated).

The implementation of the server 230 is well known to the person skilled in the art of the present technology. However, briefly speaking, the server 230 comprises a communication interface (not illustrated) structured and configured to communicate with various entities (such as the workstation computer 215, for example and other devices potentially coupled to the network 220) via the communications network 220. The server 230 further comprises at least one computer processor (e.g., a processor 110 or GPU 111 of the electronic device 100) operationally connected with the communication interface and structured and configured to execute various processes to be described herein.

In one or more embodiments, the server 230 may be implemented as the electronic device 100 or comprise components thereof, such as the processor 110, the graphics processing unit (GPU) 111, the solid-state drive 120, the random-access memory 130, the display interface 140, and the input/output interface 150.

It will be appreciated that the server 230 may provide the output of one or more processing steps to another electronic device for display, confirmation and/or troubleshooting. As a non-limiting example, the server 230 may transmit images, calculated values, results, machine learning parameters, for display on a client device configured similar to the electronic device 100 such as a smart phone, tablet, and the like.

The server 230 has access to the set of machine learning (ML) models 250.

### Machine Learning (ML) Models

The set of ML models 250 comprises inter alia a resolution enhancer ML model 260, a denoising ML model 265, a segmentation ML model 270, a flow disturbance estimation ML model 275, and a strain estimation ML model 280.

ML models will now be referred to as models.

Each of the set of models 250 is parametrized by inter alia model parameters and hyperparameters.

The model parameters are configuration variables of the model used to perform predictions and which are estimated or learned from training data, i.e. the coefficients are chosen during learning based on an optimization strategy for outputting a prediction. The hyperparameters are configuration variables of a model which determine the structure of the initial model and how the initial model is trained.

It will be appreciated that the number of model parameters to initialize will depend on inter alia the type of model (i.e., classification or regression), the architecture of the model (e.g., DNN, SVM, etc.), and the model hyperparameters (e.g. a number of layers, type of layers, number of neurons in a NN).

In one or more embodiments, the hyperparameters include one or more of: a number of hidden layers and units, an optimization algorithm, a learning rate, momentum, an activation function, a minibatch size, a number of epochs, and dropout.

### Resolution Enhancer Model

The resolution enhancer model 260 is configured to enhance the resolution of ultrasound images. It will be appreciated that the resolution enhancer model 260 may not be used in each and every embodiment of the present technology.

It is contemplated that in some embodiments of the present technology, a plurality of resolution enhancer models may accomplish the function of the resolution enhancer model 260.

In one or more embodiments, the resolution enhancer model 260 may be implemented as a very deep super-resolution (VDSR) neural network.

In one or more embodiments, the resolution enhancer model 260 learns to enhance resolution of ultrasound images to obtain a High Resolution (HR) image given a Low Resolution (LR) image. By using the HR image as a target (or ground-truth) and the LR image as an input, the resolution enhancer model 260 is trained in a supervised manner to enhance ultrasound images.

As a non-limiting example, in embodiments where the resolution enhancer model 260 is implemented as VDSR, the LR image is interpolated to obtain an interpolated low-resolution (ILR) image and input to the resolution enhancer model 260 network. The ILR image goes through (D−1) times of Convolution and Rectified Linear Unit (ReLU) layers, followed by a D-th Conv (Conv.D (Residual)) and the output is added with the ILR image and obtain the HR image.

In one or more embodiments, the ground-truth HR images may include CT and/or MRI images of the aorta of the same patient. In alternative embodiments, training may be performed with unprocessed radio-frequency (RF) signals available on some types of ultrasound apparatuses.

The resolution enhancer model 260 is configured to output HR ultrasound images based on the ultrasound image received from the medical imaging apparatus 210.

### Denoising Model

The denoising model 265 is configured to denoise ultrasound images.

It is contemplated that in some embodiments of the present technology, a plurality of ML models or networks may accomplish the function of the denoising model 265.

It will be appreciated that noise is an unwanted signal which is introduced during transmission process because of a noisy channel or due to the image acquisition process. Various types of noises which may be present in ultrasound images include one or more of Gaussian noise, salt and pepper noise, speckle noise, Poisson noise, as well as noise arising from effects of air in the intestines, and scattering from metallic portions of EVARs and other stents.

In one or more embodiments, the denoising model 265 may be implemented as a cycle-consistent generative adversarial network (CycleGAN).

The denoising model 265 may be trained to denoise 2D images so as to improve their quality (i.e., resolution and/or filtering of noise). The denoising model 265 may be trained to modify images from a source domain to a target domain. The source domain includes source noisy and/or low-quality images of an object, and the target domain includes high-quality images of the same object. The purpose of the denoising model 265 is to learn how to generate a higher quality target domain image from the source domain noisy image, i.e., transfer all the image characteristics from one image domain to another.

In one or more embodiments, the denoising model 265 comprises a generator network (or generator) and a discriminator network (or discriminator). The generator is configured to generate realistic images in the translated domain (high-quality image domain). The discriminator is configured to evaluate the generated images. The generator and discriminator may be trained simultaneously.

In one or more embodiments, the denoising model 265 may have been specifically trained to denoise 2D ultrasound images of the aorta.

In one or more embodiments, the denoising model 265 is configured to denoise HR ultrasound images having been generated by the resolution enhancer model 260. In one or more other embodiments, the denoising model 265 is configured to first denoise ultrasound images, which may be then transmitted to the resolution enhancer model 260 for transforming the denoised images into HR ultrasound images.

In one or more alternative embodiments, the denoising model 265 may be combined with the resolution enhancer model 260.

Additionally or alternatively, filtering techniques such as filtering order statistics filter, Gaussian filter, bilateral filter, mean filter, and Laplacian filter may be used.

As a non-limiting example, the denoising model 265 may be configured to remove artifacts including reflections, shadowing, and refraction from ultrasound images based on CT or MRI images of the same patient.

The denoising model 265 is configured to output denoised ultrasound images.

It will be appreciated that the denoising model 265 may not be used in each and every embodiment of the present technology, for example by determining that the noise level is below a threshold, and/or based on the apparatus model or acquisition parameters.

### Segmentation Model

The segmentation model 270 is configured to perform segmentation of tissues in ultrasound images. In one or more embodiments, the segmentation model 270 is configured to perform semantic segmentation of tissues, where the segmentation model 270 is configured to detect all borders (i.e., delimit) and discriminate (i.e., classify) various tissue types in ultrasound images of a blood vessel. The segmentation model 270 is a model having been previously trained to perform this task.

In one or more embodiments, the segmentation model 270 is configured to receive as an input HR and/or denoised ultrasound images having been output by the resolution enhancer model 260 and the denoising model 265, respectively.

The segmentation model 270 is configured to segment the outside wall of the blood vessel, the inside wall of the blood vessel, the lumen, and in some embodiments the intraluminal thrombus (ILT). Thus, for an aorta, the segmentation model 270 may classify each pixel in an ultrasound image as being one of: the outside wall of the aorta, the inside wall of the aorta, the lumen, the intraluminal thrombus (ILT) (if present), and background (e.g., pixels outside of the outside wall).

In one or more embodiments, the segmentation model 270 refers to a plurality of segmentation models 270, each configured to perform a particular segmentation task. As a non-limiting example, the segmentation models 270 may include a first segmentation model configured to perform foreground and background segmentation, a second segmentation model configured to perform semantic segmentation of lumens in aortas, and a third model configured to perform classification of pathological tissues (e.g., classification of calcified versus non-calcified tissues in the aortic wall and intraluminal thrombus (if present)). A non-limiting example of such segmentation models is described in International Patent Application No. PCT/IB2022/051558 entitled “METHOD AND SYSTEM FOR SEGMENTING AND CHARACTERIZING AORTIC TISSUES” filed on Feb. 22, 2022 by the same Applicant.

In one or more embodiments, the segmentation model 270 comprises a fully convolutional neural network (FCN).

In one or more embodiments, the segmentation model 270 has been trained to perform segmentation of aortas in ultrasound images. In one or more embodiments, the segmentation model 270 may be trained to perform segmentation based on segmented ultrasound images and corresponding segmented CT or MRI images.

In some embodiments of the present technology, the segmentation model 270 may perform segmentation further based on Doppler images acquired by the medical imaging apparatus 210.

In one or more embodiments, the segmentation model 270 has a ResNet-based FCN architecture. Non-limiting examples of ResNet include ResNet50 (50 layers), ResNet101 (101 layers), ResNet152 (152 layers), ResNet50V2 (50 layers with batch normalization), ResNet101V2 (101 layers with batch normalization), and ResNet152V2 (152 layers with batch normalization).

In one or more alternative embodiments, the segmentation model 270 may be implemented based on one of: AlexNet, GoogleNet, and VGG.

### Flow Disturbance Estimation Model

The flow disturbance estimation model 275 is configured to estimate flow disturbance in 2D ultrasound Doppler images of the aorta.

The flow disturbance estimation model 275 is configured to determine forward flow and reverse flow values at different locations in the Doppler ultrasound images during a cardiac cycle. Additionally, the flow disturbance estimation model 275 may estimate pressure.

In one or more embodiments, the flow disturbance estimation model 275 may not be a machine learning model. It will be appreciated that the Doppler signal is a direct measurement of velocity which enables identifying turbulence, low flow, reverse flow, and the like. The flow disturbance estimation model 275 may be used to apply a correction factor that may be determined after being validated after performing a computer fluid dynamics (CFD) simulation procedure (e.g., by using Ansys® Fluent® marketed by Ansys inc.) and/or MRI images.

In one or more other embodiments, the flow disturbance estimation model 275 may be trained to estimate flow disturbance in 2D ultrasound Doppler images by correlating measured local blood velocities with CFD simulation analysis having been performed using CT and/or MRI images. In such embodiments, the flow disturbance estimation model 275 may be a machine learning model based on deep neural architectures, such as, but not limited to, transformer and long short-term memory (LSTM).

During training, the flow disturbance estimation model 275 receives training datasets including, for each given patient: a set of Doppler ultrasound images, and blood flow parameters such as velocity and pressure at each of the nodes that have been generated during a computational fluid dynamic (CFD) simulation procedure. The flow disturbance estimation model 275 is then trained to estimate the blood flow values in the Doppler ultrasound images based on the blood flow values computed during the CFD simulation procedure performed on CT or MRI images of the same patient.

It will be appreciated that the blood flow parameter used for training the flow disturbance estimation model 275 may be generated during a CFD simulation procedure simulating blood flow in the arterial geometry by employing a finite volume method for the numerical implementation of the Navier-Stokes equations describing fluid flow. The CFD simulation procedure finite volume method to solve the discretized form of the Navier-Stokes equations over all the finite volume elements in the domain. The CFD simulation procedure applies an iterative approach to simulate blood flow to obtain a converged numerical solution due to the governing equations being non-linear and coupled. It should be noted that in alternative embodiments of the present technology, finite element or finite difference methods could be used instead of finite volume methods to obtain the same CFD parameters. Such a CFD simulation procedure is described in more detail in International Patent Application Publication WO 2021/059243 A1 entitled “METHOD AND SYSTEM FOR DETERMINING REGIONAL RUPTURE POTENTIAL OF BLOOD VESSEL” by the same Applicant.

The flow disturbance estimation model 275 outputs a blood flow parameter or index including blood flow velocity values and pressure values in ultrasound Doppler images.

### Database

The database 235 is configured to inter alia: (i) store acquisition parameters and data related to the medical imaging apparatus 210; (ii) store medical images including ultrasound images; (iii) store model parameters and hyperparameters of the set of ML models 250; (iv) store datasets for training, testing and validating the set of ML models 250; (v) store data output by the set of ML models 250, and (vi) store strain and/or RW parameters and/or maps.

The database 235 is configured to store ultrasound images and videos. In one or more embodiments, the database may store Digital Imaging and Communications in Medicine (DICOM) files, including for example the DCM and DCM30 (DICOM 3.0) file extensions. Additionally or alternatively, the database 235 may store medical image files in the Tag Image File Format (TIFF), Digital Storage and Retrieval (DSR) TIFF-based format, and the Data Exchange File Format (DEFF) TIFF-based format.

In one or more embodiments, the database 235 may store ML file formats, such as .tfrecords, .csv, .npy, and .petastorm as well as the file formats used to store models, such as .pb and .pkl. The database 235 may also store well-known file formats such as, but not limited to image file formats (e.g., .png, jpeg, .exif, .bmp, .tiff), video file formats (e.g., .mp4, .mkv, etc), archive file formats (e.g., .zip, .gz, .tar, .bzip2), document file formats (e.g., .docx, .pdf, .txt) or web file formats (e.g., .html).

It will be appreciated that the database 235 may store other types of data such as validation datasets (not illustrated), test datasets (not illustrated) and the like.

### Communication Network

In some embodiments of the present technology, the communications network 220 is the Internet. In alternative non-limiting embodiments, the communication network 220 can be implemented as any suitable local area network (LAN), wide area network (WAN), a private communication network or the like. It should be expressly understood that implementations for the communication network 220 are for illustration purposes only. How a communication link 225 (not separately numbered) between the medical imaging apparatus 210 and/or the server 230 and/or another electronic device (not illustrated) and the communications network 220 is implemented will depend inter alia on how each of the medical imaging apparatus 210, and the server 230 is implemented.

The communication network 220 may be used in order to transmit data packets amongst the medical imaging apparatus 210, the server 230 and the database 235. For example, the communication network 220 may be used to transmit requests between the medical imaging apparatus 210 and the server 230.

### Regional Weakening (RW) Map Generation Procedure

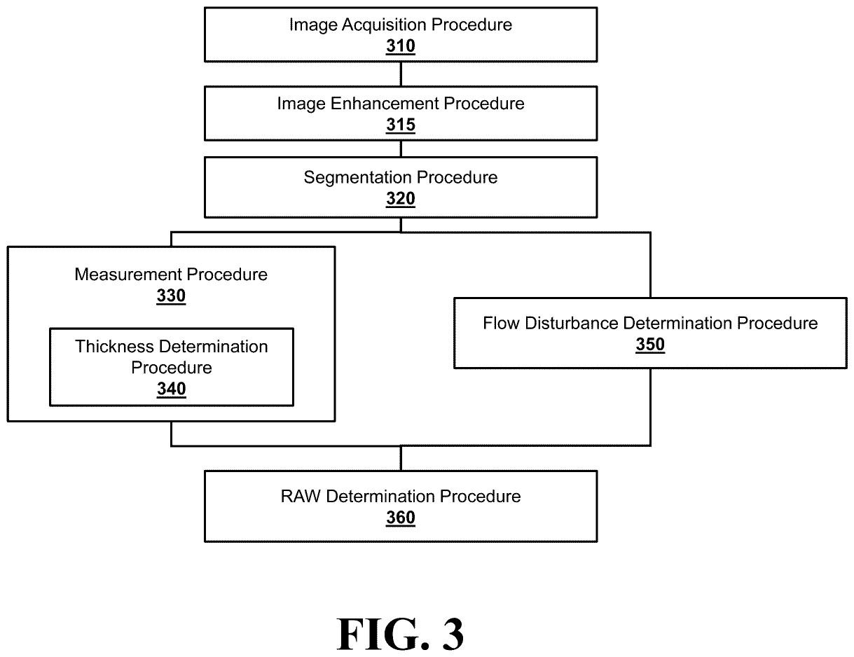

With reference to FIG. 3, there is illustrated a schematic diagram of a regional weakening (RW) map generation procedure 300 in accordance with one or more non-limiting embodiments of the present technology.

The RW map generation procedure 300 is executed by at least one electronic device such as the electronic device 100, which may be implemented as a mobile device (e.g., smartphone, tablet), as a laptop, a desktop computer, a server (e.g., server 230) or may be integrated into the medical imaging apparatus 210.

The RW map generation procedure 300 comprises inter alia an image acquisition procedure 310, an image enhancement procedure 315, a segmentation procedure 320, a measurement procedure 330, a thickness determination procedure 340, a flow disturbance determination procedure 350, and a RW determination procedure 370.

It is contemplated that portions (i.e., some procedures) of the RW map generation procedure 300 may be executed in a distributed manner (by more than one electronic device). As a non-limiting example, a first computing device may execute the image acquisition procedure 310, the image enhancement procedure 315, the segmentation procedure 320, at least a portion of the measurement procedure 330, the thickness determination procedure 340 and the flow disturbance determination procedure 350, and a second computing device may execute another portion of the measurement procedure 330 and the RW determination procedure 370.

It will be appreciated that the image enhancement procedure 315 may be optional in some embodiments of the present technology.

### Image Acquisition Procedure

The image acquisition procedure 310 is configured to inter alia: (i) receive ultrasound images from the medical imaging apparatus 210; and (ii) receive Doppler ultrasound images from the medical imaging apparatus 210.

The image acquisition procedure 310 is executed to obtain a plurality of ultrasound images during a period of time (e.g., a cardiac cycle). It will be appreciated that the ultrasound images are acquired as a sequence of frames during a time period and show movements over time (e.g., a loop). At minimum, the ultrasound images include one image at peak systole and one image at peak diastole.

The image acquisition procedure 310 may refer to ultrasound images that are generated at the medical imaging apparatus 210 via the user 202 manipulating the probe 214 of the medical imaging apparatus 210 and/or to the ultrasound images that are received by the server 230 or another processing device after having been generated at the medical imaging apparatus 210.

In one or more embodiments, the image acquisition procedure 310 may be executed online or in real-time during at least a portion of the RW map generation procedure 300. In such embodiments, the physician, technician or user 202 operating the medical imaging apparatus 210 may acquire images in real time and the images may be transmitted to and received by the server 230 (or another processing device) depending on the requirements during the RW map generation procedure 300.

The image acquisition procedure 310 is configured to receive axial ultrasound images of the blood vessel of the patient 204 acquired using the probe 214 operated by the user 202. The image acquisition procedure 310 is configured to acquire Doppler ultrasound images of the blood vessel of the patient, which are acquired from the same location as the non-Doppler ultrasound images. Similarly to the ultrasound images, the Doppler images are acquired as a sequence of frames during a period of time (e.g., cardiac cycle) in order to see the highest and lowest blood velocities throughout the cycle.

In one or more alternative embodiments, the image acquisition procedure 310 receives one static ultrasound image and M-mode (movement mode) information in place of the set of images.

It will be appreciated that the quality and quantity of images depend on the medical imaging apparatus 210, the user 202 operating the probe 214 and/or the anatomy of the given patient 204.

It will be appreciated that the blood vessel may include one or more of the aorta, iliac arteries and/or visceral arteries of the given patient 204. The aorta may include one or more of a thoracic aorta, a proximal aorta, a mid-aorta, and a distal aorta.

In one or more other embodiments, the image acquisition procedure 310 may be executed offline. The image acquisition procedure 310 may be executed after all required images of the given patient 204 are acquired at the medical imaging apparatus 210 and transmitted to the server 230 (or another processing device) without additional images of the given patient 204 being acquired thereafter.

In some embodiments of the present technology, the RW map generation procedure 300 comprises an image enhancement procedure 315.

In one or more embodiments, the image enhancement procedure 315 is executed after the image acquisition procedure 310 or concurrently with the image acquisition procedure 310 (i.e., for each acquired image in real-time).

### Image Enhancement Procedure

The image enhancement procedure 315 is configured to enhance ultrasound images received from the medical imaging apparatus 210 via the image acquisition procedure 310 so as to improve their quality, information content and resolution.

The image enhancement procedure 315 uses the resolution enhancer model 260 and the denoising model 265 of the set of models 250.

The image enhancement procedure 315 uses the resolution enhancer model 260 on one or more ultrasound images to obtain high resolution (HR) ultrasound images, and uses the denoising model 265 on the HR ultrasound image to obtain a denoised HR ultrasound image. It is contemplated that in some embodiments of the present technology, the image enhancement procedure 315 may use the denoising model 265 to denoise the ultrasound image prior to using the resolution enhancer model 260 to obtain an HR image.

In one or more embodiments, a sufficient resolution and/or signal may be determined experimentally. It will be appreciated that resolution in ultrasound may be difficult to define. As a non-limiting example, the axial resolution of a 5 MHz ultrasound scan is 0.3 mm; a 2 MHz transducer is 0.8 mm. However, the lateral resolution depends on depth as it decreases with deeper structures and depends on how well the transducer is focused. Temporal resolution is typically 10-30 fps. It is contemplated that any resolution that is acceptable to a qualified clinician may be used as in the majority of cases, which will generally be better resolution than obtained from CT. Signal quality will generally not be limited by the resolution. It will be limited by noise, poor skin contact, poor adjustment of gain or focus, lack of coupling medium, presence of intestinal gas, poor handling of the transducer resulting in images acquired at an angle instead of at 90 degrees, and images where the entire diameter is not visible due to operator error. In some embodiments, ultrasound images may be assessed to determine if signal quality is sufficient (e.g., above a threshold, which may be determined experimentally).

Additionally, the image enhancement procedure 315 may also use pre-processing and post-processing techniques to improve the quality of the images.

The image enhancement procedure 315 may for example use filtering techniques. Non-limiting examples of filtering techniques include filtering order statistics filter, Gaussian filter, bilateral filter, mean filter, and Laplacian filter may be used.

In some embodiments of the present technology, the image enhancement procedure 315 may be executed in real-time or almost real-time such that the HR denoised image may be displayed to the user 202 operating the medical imaging apparatus 210 (e.g., on a display of the medical imaging apparatus 210 or display of a user device).

In one or more other embodiments, the image enhancement procedure 315 may be executed offline (i.e., after required ultrasound images have been acquired).

The image enhancement procedure 315 outputs HR and/or denoised ultrasound images of the blood vessel.

The RW map generation procedure 300 comprises a segmentation procedure 320 which may be optional in some embodiments of the present technology, for example when segmentation is performed by the user 202 visually using electronic calipers on the medical imaging apparatus 210.

### Segmentation Procedure

The segmentation procedure 320 is configured to inter alia: (i) receive ultrasound images; (ii) segment, using the segmentation model 270, the ultrasound images to obtain segmented tissues of the blood vessel.

The segmentation procedure 320 is used for inter alia detecting and delimiting different blood vessel tissues in the ultrasound images such that their composition, position, and movement may be assessed.

In one or more embodiments, the segmentation procedure 320 receives HR denoised images from the image enhancement procedure 315.

The segmentation procedure 320 uses the segmentation model 270 to segment (i.e. classify each pixel) aortic tissues in ultrasound images acquired by the medical imaging apparatus 210. It will be appreciated that the segmentation procedure 320 may use one or more segmentation ML models 270 having been trained to segment tissues in images of the blood vessel.

In one or more embodiments, the segmentation procedure 320 may be executed in real-time, i.e., while the user 202 operates the probe 214 of the medical imaging apparatus 210 such that the segmented tissues are displayed on a display screen of the medical imaging apparatus 210.

In one or more embodiments, the segmentation procedure 320 may segment tissues based on specific images which depend on the acquisition angle and parameters of the probe 214. For example, the segmentation procedure 320 may perform segmentation based on image scanning along the entire aorta, and by measuring both static and dynamic images. As stated herein above, the segmentation model 270 may have been trained to perform segmentation using CT and/or MRI images of the aorta in addition to ultrasound images of the aorta.

In one or more embodiments, the segmentation procedure 320 may use the ultrasound Doppler images to perform segmentation.

Additionally, in some embodiments, the user 202 may be able to confirm whether the segmentation of the tissues is accurate and/or may correct the segmentation results. In such embodiments, the segmentation model 270 may be continuously trained to provide more accurate results based on the input of the user 202.

In one or more alternative embodiments, the segmentation procedure 320 may perform segmentation based on user inputs from the user 202 operating the medical imaging apparatus 210. As a non-limiting example, the user 202 may provide indications via an input/output interface of the medical imaging apparatus 210 (or via another computing device, e.g., smartphone) to assist the segmentation model 270 in performing segmentation.

In some embodiments, such as but not limited to embodiments where the segmentation procedure 320 is performed in real-time, the segmentation procedure 320 may color borders (edges) of the different segmented tissues, which may be displayed on a display screen of the medical imaging apparatus 210 or of another electronic device.

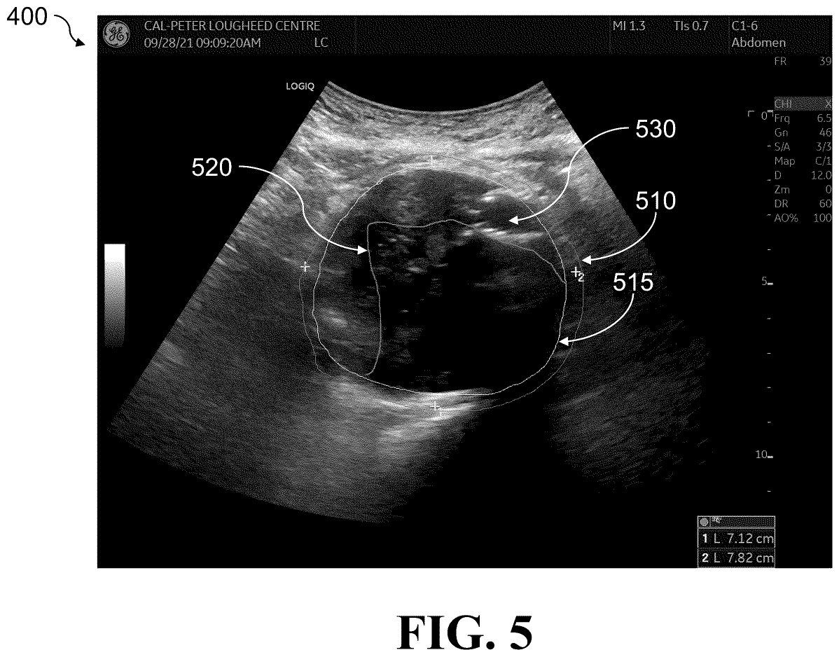

FIG. 5 illustrates a non-limiting example of segmentation in an ultrasound image 400, which shows borders of the segmented outside wall 510, borders of the segmented inside wall 515, borders of the segmented lumen 520, and a thrombus 530 present within the lumen between borders of the segmented inside wall 515, borders of the segmented lumen 520.

The segmentation procedure 320 outputs one or more segmented ultrasound images. The segmentation procedure 320 outputs segmented tissues including outside wall, inside wall, lumen and thrombus (if present).

In one or more embodiments, the segmentation procedure 320 outputs indications (i.e., coordinates) of delimitations of the segmented tissues in the ultrasound images. As a non-limiting example, the segmentation procedure 320 may output a plurality of edge (border) coordinates for each segmented tissue.

### Measurement Procedure

The measurement procedure 330 is configured to inter alia: (i) receive segmented ultrasound images; (ii) measure, based on the segmented ultrasound images over time, areas of interest in the blood vessel; and (iii) measure movements in the areas of interest of the blood vessel.

The measurement procedure 300 is configured to determine, for each ultrasound image, measurements related to the segmented tissues in the blood vessel, such that movement of each of the segmented tissues may be quantified. The movement of the segmented tissues in the blood vessel are indicative of deformation and strain and will be used to assess regional weakening in the blood vessel.

In one or more embodiments, the measurement procedure 330 receives segmented images from the segmentation procedure 320. It will be appreciated that the segmented ultrasound images may be segmented HR ultrasound images having been segmented after the image enhancement procedure 315.

In one or other embodiments, the measurement procedure 330 may be performed automatically with minimal user intervention. In such embodiments, the measurement procedure 330 may use one or more of the set of ML models 250 such as the segmentation model 270 to segment tissues (e.g., inside wall, outside wall, and lumen) and measure areas of interest (e.g., edges or borders) in the segmented tissues in each image taken in time. It will be appreciated that the measurement procedure 330 may be combined with the segmentation procedure 320 such that for a given segmented tissue in an image, differences in the given segmented tissue over time (i.e., compared other images taken during the period of time) are automatically calculated.

In one or more other embodiments, the measurement procedure 330 may be performed semi-automatically upon receiving inputs by the user 202 operating the medical imaging apparatus 210. As a non-limiting example, the user 202 may indicate, via an input/output interface, location(s) (i.e., coordinates or points) in the ultrasound images to perform the measurements. In one or more embodiments, the measurement procedure 330 automatically measures and reports the measurements to the user 202 operating the medical imaging apparatus 210 for validation.

In one or more embodiments, the measurement procedure 330 may be executed directly by a processing device of the medical imaging apparatus 210 and its output provided to the RW map generation procedure 300. In one or more other embodiments, the measurement procedure 330 may be executed by another processing device such as the workstation computer (not illustrated), the server 230, a mobile device (not illustrated) and the like.

In one or more embodiments, the measurement procedure 330 is configured to measure, by receiving user inputs, one or more of: a supraceliac abdominal aorta diameter, a suprarenal abdominal aorta diameter, an infrarenal abdominal aorta diameter, and an illiac diameter.

In one or more alternative embodiments, the measurement procedure 330 is configured to measure the internal diameters (i.e., inner anterior wall to the inner posterior wall (inner to inner or ITI) or intima to intima) and/or measure the external diameters (i.e., from the outer anterior to the outer posterior wall (OTO) or adventitia to adventit) and/or perform leading edge to leading edge measurements.

In one or more embodiments, the measurement procedure 330 determines a maximum transverse diameter of the aneurysmal sac, a longitudinal length, shape measures (saccular/fusiform/eccentric), upper extent measure relative to the renal arteries, lower extent measure, including extension into any branches, any side or visceral branches arising from the aneurysm.

The measurement procedure 330 is configured to perform measurements related to one or more of the outer wall, inner wall, lumen, and thrombus. It will be appreciated that the measurement procedure 330 may perform a plurality of measurements in each image for each of the outer wall, inner wall, lumen, and thrombus (if present). The plurality of measurements of the outer wall, inner wall, lumen, and thrombus enable assessing their properties and determining movement of the aforementioned tissues over time.

In one or more embodiments, the measurement procedure 330 outputs a set of diameters relating to the movement of tissues of the blood vessel over time.

The measurement procedure 330 is configured to determine, by analyzing and comparing the measurements in each image taken over time (e.g., frame) minimum, maximum, and equivalent circle diameters and eccentricity for the segmented tissues.

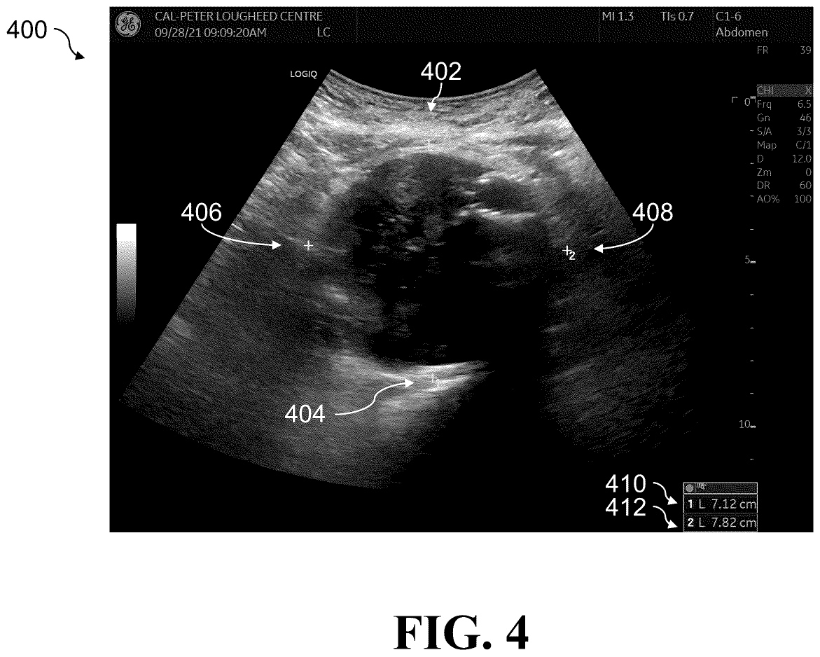

FIG. 4 illustrates a non-limiting example of an axial ultrasound image 400 of a body of a patient with four radial points 402, 404, 406, 406 along the circumference of the aorta of the patient. The axial ultrasound image 400 includes diameter measurements at the bottom right: a first diameter 410 of 7.12 cm (i.e., between points 402 and 404) and a second diameter of 7.82 cm (i.e., between points 402 and 404).

In one or more embodiments, the measurement procedure 330 determines the radial deformation by analyzing the set of ultrasound images taken over time based on the set of measurements. The measurement procedure 330 determines variation of the borders (i.e. radial points) of the segmented tissues by comparing the ultrasound images taken over time to obtain the radial deformation of the segmented tissues.

The radial deformation provides an indication of strain in the blood vessel. The measurement procedure 330 outputs a radial deformation index or parameter including radial deformation values in regions of the blood vessel, which are at least partially indicative of strain.

In one or more embodiments, the measurement procedure 330 may use a ML model (not illustrated) or heuristics to calculate strain values based on radial deformation values.

In some embodiments, prior to measuring the deformation and thrombus load, the measurement procedure 330 transmits the ultrasound images to the segmentation procedure 320 to obtain a clear delimitation of the aortic walls, lumen, and thrombus (if present). The measurement procedure 330 then measures and determines the deformation values of the walls and lumen.

The measurement procedure 330 outputs radial deformation values.

The measurement procedure 330 comprises a thickness determination procedure 340.

### Thickness Determination Procedure

The thickness determination procedure 340 is configured to measure thrombus load or thickness based on a distance between the lumen and an inside wall of the blood vessel. The thrombus load corresponds to intraluminal thrombus (ILT) thickness measurements.

It will be appreciated that in some embodiments, there may be no thrombus present in the aorta and thus the measured thrombus load may be equal to 0.

In one or more embodiments, the thickness determination procedure 340 receives segmented ultrasound images from the segmentation procedure 320 and determines, using the segmented ultrasound images, the thrombus load based on a distance between the lumen and an inside wall of the aorta.

In one or more embodiments, the thickness determination procedure 340 measures the thrombus load based on measurements performed during the measurement procedure 330.

It will be appreciated that the thickness determination procedure 340 may determine the distance based on the coordinates of the borders of the segmented lumen and the segmented inside wall of the aorta. The distance may be measured at each radial point. It will be appreciated that the measured distance may be averaged to obtain the thrombus load.

In one or more embodiments, if there is enough resolution to discriminate between the interface between the thrombus surface and the interior surface of the wall, the thickness determination procedure 340 determines a thrombus load.

In one or more embodiments, the thrombus load is measured from the interior surface of the artery to the edge of the thrombus. If the wall of the artery is very thin, or the wall cannot be segmented, the thrombus load may be measured from the exterior surface of the artery wall to the edge of the thrombus.

The thickness determination procedure 340 outputs the thickness parameter including thrombus thickness values.

The flow disturbance determination procedure 350 may be performed at any time prior to the RW determination procedure 360 and concurrently with other procedures.

### Flow Disturbance Determination Procedure

The flow disturbance determination procedure 350 is configured to determine blood flow disturbance based on the 2D ultrasound Doppler images.

The flow disturbance determination procedure 350 determines flow disturbance values directly from Doppler signal. The Doppler images taken over a period of time (e.g., a cardiac cycle) include the highest and lowest velocities throughout the period of time.

In one or more embodiments the flow disturbance determination procedure 350 determines, based on the Doppler images, turbulence, low flow, and reverse flow.

As explained herein above, in alternative embodiments, the flow disturbance estimation model 275 may have been previously trained to estimate flow disturbance in 2D ultrasound Doppler images by correlating measured local blood velocities with a CFD simulation analysis having been performed using CT or MRI images.

The flow disturbance determination procedure 350 outputs a blood flow parameter or blood flow measurements including forward flow and reverse flow values at different locations in the Doppler ultrasound images during a period of time (e.g., cardiac cycle).

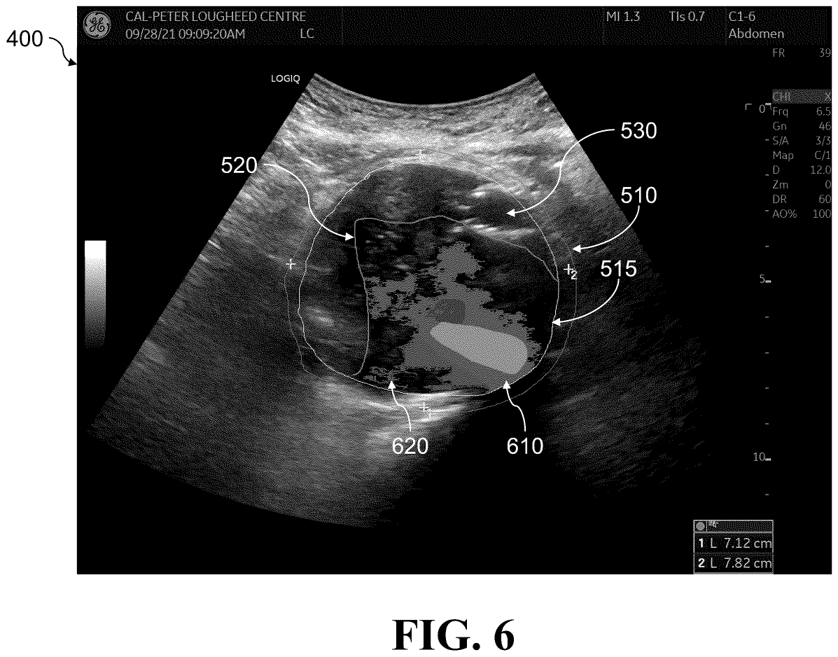

FIG. 6 depicts a simulated Doppler image showing forward flow 610 and reverse flow 620. The Doppler image will inform the image enhancement process regarding the segmentation of the lumen and will also reveal flow disturbances that are used to calculate the RW index.

### RW Determination Procedure

The RW determination procedure 360 is configured to inter alia: (i) receive one or more ultrasound images; (ii) receive the thickness measurements, flow disturbance measurements, and deformation measurements; (iii) generate, based on the thickness measurements, and the deformation measurements, a radial strain parameter comprising radial strain values; (iv) generate, based on the thickness measurements, the flow disturbance measurements, and the deformation measurements, and the radial strain parameter, a regional weakening (RW) parameter comprising regional weakening values; (v) generate, based on the radial strain parameter and the ultrasound images, a radial strain map of the circumference of the wall of the blood vessel; and (vi) generate, based on the radial strain parameter, the RW parameter and the ultrasound images, a RW map of the circumference of the wall of the blood vessel.

The RW determination procedure 360 receives radial deformation measurements from the measurement procedure 330, blood flow disturbance measurements from the flow disturbance determination procedure 350, and thickness measurements from the thickness determination procedure 340.

In one or more embodiments, the RW determination procedure 360 determines local material stiffness based on strain and blood pressure. It will be appreciated that in this context, the radial deformation measurements and the blood flow velocities may be used as proxies for strain and blood pressure.

In one or more other embodiments, the user 202 may assess local material stiffness which may be used to refine RW measurements.

The RW determination procedure 360 determines radial strain values based on the thickness measurements and the deformation measurements, a radial strain parameter comprising radial strain values. The RW determination procedure 360 generates a radial strain parameter or index comprising radial strain values at locations along a circumference of the blood vessel.

The RW determination procedure 360 determines regional weakening (RW) values based on the radial deformation values, blood flow disturbance values and the thrombus thickness values. The RW determination procedure 360 generates a RW parameter or index comprising the RW values at locations along a circumference on the blood vessel.

In one or more embodiments, the RW determination procedure 360 may show segments of the circumference of the blood vessel, or show a continuous distribution around the circumference. It will be appreciated that the diameter is measured at 90 degrees to the patient torso (with some error due to the inability of the operator to hold the transducer at exactly 90 degrees).

The RW determination procedure 360 determines a RW parameter or index including RW values. The RW parameter is representative of a state of regional weakening and probabilities of rupture of a region or a set of regions of a blood vessel wall. The RW parameter takes into account different factors to adverse remodeling and degeneration of a vessel wall, including but not limited to the wall, and is indicative of a localized state of weakening of the blood vessel and consequent expansion and rupture potential.

The RW determination procedure 360 displays RW values on the ultrasound images. It will be appreciated that that the ultrasound images may be the HR denoised ultrasound images, and the RW determination procedure 360 may display the RW values around the circumference of the blood vessel.

In one or more embodiments, the RW determination procedure 360 displays strain and RW scores in segments around the circumference of the image. As a non-limiting example there may be twelve (12) patches, but it will be appreciated that the size and number of the patches may vary. It is contemplated that in some embodiments, there could also be a continuous display of strain and RW instead of using patches.

In one or more embodiments, a temporal resolution above a threshold might allow assessment of viscoelasticity in the wall and in the thrombus (if present). Viscoelastic properties, in particular energy loss have been linked to wall degeneration in the thoracic aorta (see paper by Chung et al. (2014) and it was shown that high RAW corresponds to higher energy loss in the abdominal aorta, see Forneris et al. (2021)).

It will be appreciated that the temporal resolution depends on the maximum depth that is being measured—ultrasound has a velocity within tissue, and the time defined by this velocity (allowing for both transmission and reception of the echo) limits the temporal resolution. The temporal resolution may be further limited by the quality of the electronics of the medical imaging apparatus 210.

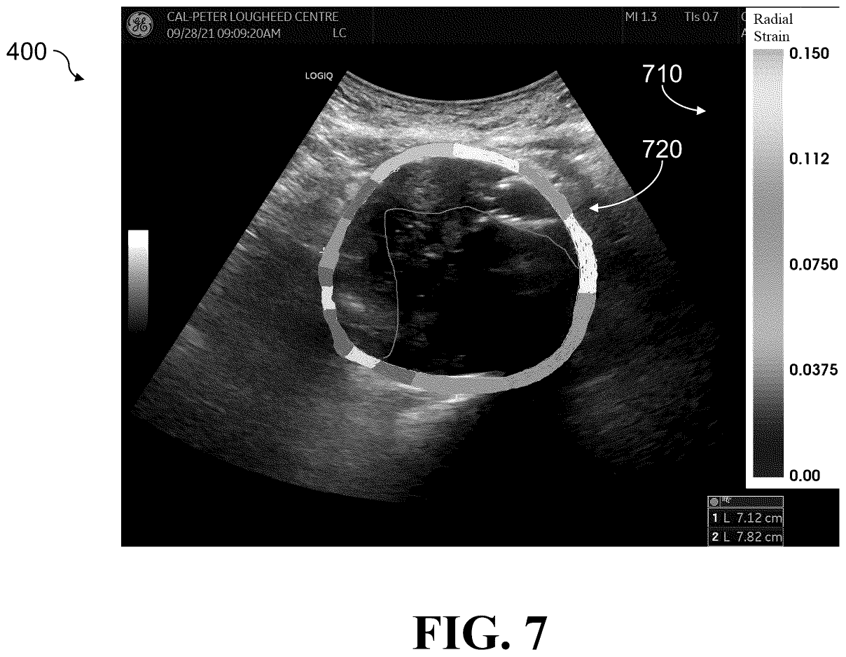

With reference to FIG. 7 there is illustrated a radial strain map 720 on the wall of the aorta superimposed on the ultrasound image 400 with radial values scale 710 on the right in accordance with one or more non-limiting embodiments of the present technology.

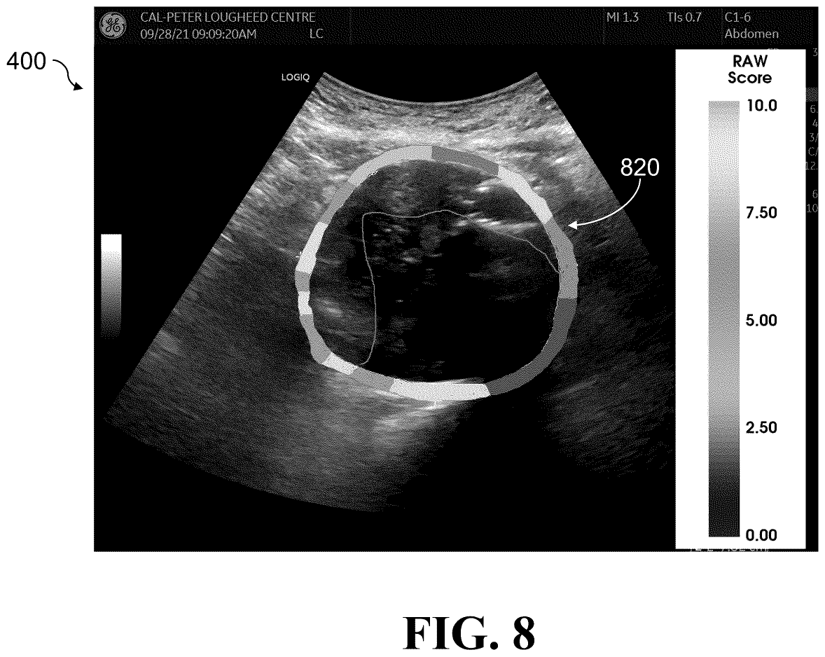

With reference to FIG. 8 there is illustrated a RAW map 720 of the circumference of the wall of the aorta superimposed on the ultrasound image 400 with a RW values scale. The RW map 720 is determined based on the deformation values, flow disturbance values, and thrombus thickness values at every point around the circumference of the wall of the aorta.

### Method Description

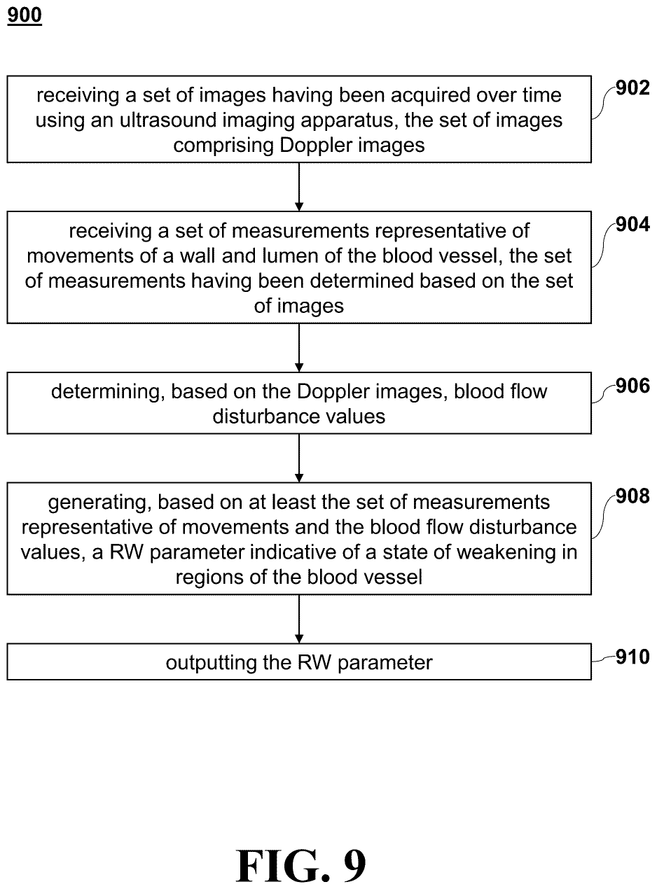

FIG. 9 depicts a flowchart of a method 900 of determining regional weakening of a blood vessel based on ultrasound images of a body of a patient 204 in accordance with one or more non-limiting embodiments of the present technology.

In one or more embodiments, the method 900 is executed by a processing device such as the processor 110 and/or the GPU 111 operatively connected to a non-transitory computer-readable storage medium such as the solid-state drive 120 and/or the random-access memory 130 storing computer-readable instructions. The processing device, upon executing the computer-readable instructions, is configured to or operable to execute the method 900.

It will be appreciated that the processing device may be the processing device of the medical imaging apparatus 210, the server 230, and/or another computerized device such as a mobile device (smartphone or laptop), a desktop computer, a laptop computer, and the like.

The processing device has access to the set of ML models 250.

The method 900 begins at processing step 902.

According to processing step 902, the processing device receives a set of images of a body of the given patient 204 having been acquired using the medical imaging apparatus 210, the set of images having been acquired over a period of time (e.g., a cardiac cycle). The medical imaging apparatus 210 is implemented as a 2D ultrasound imaging apparatus.

The set of images include ultrasound images and Doppler images taken over a period of time. The set of images includes axial ultrasound images of a portion of the body of the patient 204. The ultrasound images of the portion of body of the patient 204 comprise at least one blood vessel. The blood vessel may include, as a non-limiting example, the aorta and/or iliac arteries. The aorta may include one or more of a thoracic aorta, a proximal aorta, a mid-aorta, and a distal aorta.

In one or more embodiments, the processing device receives a plurality of ultrasound images. The processing device accesses the resolution enhancer model 260 and the denoising model 265 of the set of models 250. The processing device uses the resolution enhancer model 260 on the plurality of ultrasound images to obtain high resolution (HR) ultrasound images and uses the denoising model 265 on the HR ultrasound image to obtain the set of images, the set of images comprising a plurality of denoised HR ultrasound image of higher quality than the plurality of ultrasound images received from the medical imaging apparatus 210. In one or more embodiments, the trained resolution enhancing ML model comprises a very deep super-resolution (VDSR) neural network, and the trained denoising ML model comprises a cycle-consistent generative adversarial network (CycleGAN).

In one or more other embodiments, the processing device receives a plurality of ultrasound images corresponding to the set of images from the medical imaging apparatus 210.

According to processing step 904, the processing device receives set of measurements representative of movements of a wall and lumen of the blood vessel, the set of measurements having been determined based on the set of images.

In one or more embodiments, prior to executing processing step 904, the processing device receives ultrasound images comprising coordinates on tissues of the blood vessel such as outside wall, inside wall, lumen and thrombus (if present). As a non-limiting example the coordinates on tissues of the blood vessel may include diameters of outside wall, inside wall, and lumen.

The processing device calculates movements of the tissues by analyzing the coordinates in the tissues in the ultrasound images over time to obtain the set of measurements representative of movements of the wall and lumen of the blood vessel.

In one or more embodiments, prior to executing processing step 904, the processing device accesses one or more trained segmentation ML models 270 to segment (i.e. classify each pixel) blood vessel tissues in ultrasound images. The trained segmentation ML models 270 may output segmented tissues including outside wall, inside wall, lumen, thrombus (if present) as well as background pixels. The segmented tissues may include locations of all pixels on edges and borders.

The processing device then determines diameter of the segmented outside wall, a diameter of the segmented inside wall, and a diameter of the segmented lumen. In one or more alternative embodiments, the user 202 may perform the measurements and segmentation manually (i.e., using a caliper) and input the measurements at the medical imaging apparatus 210 (or another electronic device) such that the measurements are received by the processing device.

The processing device is configured to perform diameter measurements of one or more of the outer wall of the aorta, the inner wall of the aorta, and the lumen. In one or more embodiments, the processing device may perform at least a portion of the measurements based on inputs from the user 202. In one or more other embodiments, the processing device may perform the diameter measurements automatically.

It will be appreciated that the processing device may perform a plurality of measurements for each of the outer wall, inner wall, lumen, and thrombus (if present).

In one or more embodiments, the processing device is configured to determine minimum, maximum, and equivalent circle diameters and eccentricity for each of the diameter measurements. It will be appreciated that the minimum, maximum, and equivalent circle diameters and eccentricity for each of the diameter measurements may be determined by the processor based on user inputs from the user 202 having been performed at the medical imaging apparatus 210.

In one or more embodiments,, the processing device measures thrombus load or thickness. The thrombus load corresponds to the intraluminal thrombus (ILT) thickness. In one or more embodiments, the thrombus load is measured from the interior surface of the artery to the edge of the thrombus. If the wall of the artery is very thin, or the wall cannot be segmented, the thrombus load is measured from the exterior surface of the artery wall to the edge of the thrombus.

It will be appreciated that if the thrombus load is equal to 0 it is indicative of an absence of thrombus.

The processing device thus obtains radial deformation values in the blood vessel, which are indicative of strain in the blood vessel. In one or more embodiments, the processing device generates a radial strain parameter or index based on at least the radial deformation values.

In one or more embodiments, the processing device may correct the radial deformation values or calculate actual strain for example by using a ML model having been trained therefor.

According to processing step 906, the processing device determines, based Doppler image in the set of images, blood flow disturbance values in the blood vessel.

In one or more embodiments, the processing device analyzes the Doppler images over time, which each include blood flow velocity values, to determine blood flow disturbance values.

In one or more alternative embodiments, the processing device accesses a trained flow disturbance estimation model 275. The flow disturbance estimation model 275 has been previously trained to estimate flow disturbance in 2D ultrasound Doppler images by correlating measured local blood velocities with fluent analysis having been performed using CT or MRI images.

The processing device outputs a blood flow parameter or blood flow values including forward flow and reverse flow values at different locations in the Doppler ultrasound images during a cardiac cycle, as well as pressure values.

According to processing step 908, the processing device generates, based on at least the set of measurements representative of movements in the blood vessel and the blood flow disturbance values, a regional aortic weakening (RW) parameter indicative of a state of weakening in regions of the aorta.

In one or more embodiments, the processing device generates, based on the radial deformation values which are indicative of strain, the thrombus thickness and the blood flow disturbances values, the RW parameter.

In one or more embodiments, the processing device defines a plurality of patches on the vessel geometries comprising the outer wall and the lumen, perpendicularly to the lumen centerline, and determines a patch-averaged distribution for each of the radial deformation values, blood flow disturbance values and the thickness values.

The processing device determines the RW parameter or index. The RW parameter is representative of a state of regional weakening and probabilities of rupture of a region or a set of regions of a blood vessel wall. The RW parameter takes into account different factors to adverse remodeling and degeneration of a vessel wall, including but not limited to the aortic wall, and is indicative of a localized state of weakening of the blood vessel and consequent expansion and rupture potential.

At processing step 910, the processing device outputs the RW parameter. In one or more embodiments, the processing device outputs a radial strain parameter with the RW parameter.

In one or more embodiments, the processing device generates a strain map based on the strain parameter and a regional weakening map based on the RW parameter using the set of ultrasound images.

In one or more embodiments, the processing device causes display of strain and RW scores in patches around the circumference of the ultrasound images.

In one or more embodiments, the processing device determines local material stiffness based on strain and blood pressure.

The method 900 then ends.

In some cases, what are believed to be helpful examples of modifications to the present technology may also be set forth. This is done merely as an aid to understanding, and, again, not to define the scope or set forth the bounds of the present technology. These modifications are not an exhaustive list, and a person skilled in the art may make other modifications while nonetheless remaining within the scope of the present technology. Further, where no examples of modifications have been set forth, it should not be interpreted that no modifications are possible and/or that what is described is the sole manner of implementing that element of the present technology.

Modifications and improvements to the above-described implementations of the present technology may become apparent to those skilled in the art. The foregoing description is intended to be exemplary rather than limiting.

1. A method for determining a regional weakening parameter of a blood vessel of a given patient, the method being executed by at least one processing device, the method comprising:
receiving a set of images of a portion of a body of the given patient having been acquired using an ultrasound imaging apparatus, the set of images comprising Doppler images;
receiving a set of measurements representative of movements of a wall and lumen of the blood vessel, the set of measurements having been determined based on the set of images;
determining, based on the Doppler images in the set of images, blood flow disturbance values in the blood vessel;
generating, based on at least the set of measurements and the blood flow disturbance values, the regional weakening parameter indicative of a state of weakening in regions of the blood vessel; and
outputting the regional weakening parameter.

2. The method of claim 1, further comprising, prior to said generating, based on at least the set of measurements and the blood flow disturbance values, the regional weakening parameter indicative of a state of weakening in regions of the blood vessel:
determining, by analyzing the set of images based on the set of measurements, a radial deformation in the blood vessel, the radial deformation being at least partially indicative of strain in the blood vessel; and
wherein said generating the regional weakening parameter is based on the radial deformation in the blood vessel.

3. The method of claim 1, further comprising, after said receiving the set of images:
detecting, for the set of images, respective wall and lumen positions in the blood vessel; and
calculating, based on the respective wall and lumen positions, a movement of the wall and the lumen of the blood vessel to thereby receive the set of measurements.

4. The method of claim 3, wherein said calculating, based on the respective wall and lumen positions, the movement of the wall and the lumen of the blood vessel comprises:
determining, based on the positions of the wall and the lumen, a thrombus thickness; and
wherein said generating the regional weakening parameter is based on the thrombus thickness.

5. (canceled)

6. (canceled)

7. The method of claim 1, further comprising:
generating a regional weakening map based on the regional weakening parameter and the set of images of the blood vessel; and
outputting the regional weakening map, the regional weakening map showing a circumference of the blood vessel comprising regional weakening values.

8. The method of claim 3 wherein said detecting, for the set of images, respective wall and lumen positions in the blood vessel comprises:
segmenting, using a trained segmentation model on the set of images, an outside wall, an inside wall, and the lumen of the blood vessel.

9. The method of claim 8, further comprising:
determining minimum, maximum, equivalent circle diameters and eccentricity of each of an outside wall diameter, an inside wall diameter and a lumen diameter.

10. (canceled)

11. The method of claim 1, further comprising, prior to said receiving the set of images of the blood vessel:
receiving, a plurality of images of the given patient having been acquired using the ultrasound imaging apparatus, the plurality of images including at least one Doppler image;
generating, using a trained resolution enhancing machine learning (ML) model on the plurality of images, a plurality of high resolution (HR) images of the blood vessel, a resolution of the plurality of HR images being above a resolution of the plurality of images; and
denoising, using a trained ML denoising model, the plurality of HR images to obtain the set of images.

12. The method of claim 11, wherein the trained resolution enhancing ML model comprises a very deep super-resolution (VDSR) neural network; and
wherein the trained denoising ML model comprises a cycle-consistent generative adversarial network (CycleGAN).

13. The method of claim 2, further comprising:
generating a strain map based on the radial deformation values and the set of images of the blood vessel; and
outputting the strain map, the strain map showing a circumference of the blood vessel comprising strain values.

14.-17. (canceled)

18. A system for determining regional weakening (RW) of a blood vessel of a given patient, the system comprising:
at least one processing device; and
a non-transitory storage medium operatively connected to the at least one processing device, the non-transitory storage medium storing computer-readable instructions;
the at least one processing device, upon executing the computer-readable instructions, being configured for:
receiving a set of images of a portion of a body of the given patient having been acquired using an ultrasound imaging apparatus, the set of images comprising Doppler images;
receiving a set of measurements representative of movements of a wall and lumen of the blood vessel, the set of measurements having been determined based on the set of images;
determining, based on the Doppler images in the set of images, blood flow disturbance values in the blood vessel;
generating, based on at least the set of measurements and the blood flow disturbance values, a regional weakening parameter indicative of a state of weakening in regions of the blood vessel; and
outputting the regional weakening parameter.

19. The system of claim 18, wherein the at least one processing device is further configured for, prior to said generating, based on at least the set of measurements and the blood flow disturbance values, the regional weakening parameter indicative of a state of weakening in regions of the blood vessel:
determining, by analyzing the set of images based on the set of measurements, a radial deformation in the blood vessel, the radial deformation being at least partially indicative of strain in the blood vessel; and
wherein said generating the regional weakening parameter is based on the radial deformation in the blood vessel.

20. The system of claim 18, wherein the at least one processing device is further configured for, after said receiving the set of images:
detecting, for the set of images, respective wall and lumen positions in the blood vessel; and
calculating, based on the respective wall and lumen positions, a movement of the wall and the lumen of the blood vessel to thereby receive the set of measurements.

21. The system of claim 20, wherein
said calculating, based on the respective wall and lumen positions, the movement of the wall and the lumen of the blood vessel comprises:
determining, based on the positions of the wall and the lumen, a thrombus thickness; and
wherein said generating the regional weakening parameter is based on the thrombus thickness.

22. (canceled)

23. (canceled)

24. The system of claim 18, wherein the at least one processing device is further configured for:
generating a regional weakening map based on the regional weakening parameter and the set of images of the blood vessel; and
outputting the regional weakening map, the regional weakening map showing a circumference of the blood vessel comprising regional weakening values.

25. The system of claim 20, wherein said detecting, for the set of images, respective wall and lumen positions in the blood vessel comprises:
segmenting, using a trained segmentation model on the set of images, an outside wall, an inside wall, and the lumen of the blood vessel.

26. The system of claim 25, wherein the at least one processing device is further configured for:
determining minimum, maximum, equivalent circle diameters and eccentricity of each of an outside wall diameter, an inside wall diameter and a lumen diameter.

27. (canceled)

28. The system of claim 18, wherein the at least one processing device is further configured for, prior to said receiving the set of images of the blood vessel:
receiving, a plurality of images of the given patient having been acquired using the ultrasound imaging apparatus, the plurality of images including at least one Doppler image;
generating, using a trained resolution enhancing machine learning (ML) model on the plurality of images, a plurality of high resolution (HR) images of the blood vessel, a resolution of the plurality of HR images being above a resolution of the plurality of images; and
denoising, using a trained ML denoising model, the plurality of HR images to obtain the set of images.

29. The system of claim 28, wherein
the trained resolution enhancing ML model comprises a very deep super-resolution (VDSR) neural network; and
wherein the trained denoising ML model comprises a cycle-consistent generative adversarial network (CycleGAN).

30. The system of claim 18, further comprising:
generating a strain map based on the radial deformation values and the set of images of the blood vessel; and
outputting the strain map, the strain map showing a circumference of the blood vessel comprising strain values.

31.-34. (canceled)
